(19) United States
(12) Patent Application Publication
(10) Pub. No.: US 2005/0220900 A1
(43) Pub. Date: Oct. 6, 2005

(54) Use of extracts containing phytoestrogen selectively modulating estrogen-receptor-beta

(76) Inventors: Michael Popp, Lauf (DE); Wolfgang Wuttke, Bovenden (DE); Hubertus Jarry, Neu-Eichenberg (DE); Volker Christoffel, Buchberg (DE); Barbara Spengler, Neumarkt/Opf. (DE)

Correspondence Address: CATALYST LAW GROUP, APC, 9710 SCRANTON ROAD, SUITE S-170, SAN DIEGO, CA, 92121, US

(21) Appl. No.: 10/505,031

(22) PCT Filed: Feb. 10, 2003
(86) PCT No.: PCT/EP03/01311

§ 371 Date: Nov. 23, 2004

(30) Foreign Application Priority Data
Feb. 15, 2002 (DE) 102 06 390.7

(51) Int. Cl. A61K 35/78

(52) U.S. Cl. 424/725

(57) ABSTRACT

The present invention relates to the use of phytoestrogen-containing extracts that selectively modulate estrogen receptor beta (ER-beta) without producing any uterotropic effect, for the treatment of clinical situations and pathophysiological conditions that are selected from the group consisting of: Obesity and thereby to possibly influence the metabolic syndrome, particularly hypertension, asterosclerosis, cardiac infarct, hyperandrogenemia; Menopausal continence disorders; Menopausal heat surges associated with hyperstimulation of the hypothalamic gonadotropin releasing hormone pulse generator; steroid hormonal synthesis disorder, particularly that of progesterone synthesis of the human corpus luteum, resulting in luteal insuffeciency; Alzheimer's disease associated with elevated expression of estrogen receptor beta in hippocampal neurons.

The present invention relates to the use of phytoestrogen-containing extracts that selectively modify estrogen receptor beta (ER-beta) without producing any uterotropic effect, according to the characterizing clause of claim 1.

Extracts from vitex agnus castus (monk's pepper, chaste tree) have been used for a long time to treat irregular periods, mastodynia, premenstrual syndrome, and abnormal menstrual bleeding (secondary to primary or secondary corpus luteum insufficiency), but these extracts have not been used in estrogen replacement therapy.

A decrease in estradiol level occurs during menopause secondary to the termination of ovarian function. This produces a reduction in the proliferative processes and leads to increased activity of the GnRH pulse generator in the hypothalamus. (The gonadotropin releasing hormone pulse generator is a type of clock in the hypothalamus and times the pulsatile secretion of LH, with the amplitude and frequency influenced by steroids.) In the menopausal woman, the resulting stimulated secretion of LH leads to perceived disruptive and increasing heat surges, the so-called “hot flashes.”

In the absence of a sufficiently high serum level of estradiol, osteoblast activity and thereby a loss of bone mass become dominant, accompanied by an elevated risk of skeletal fractures. A long term risk of plaque development in the vascular system appears concomitantly and brings with it an elevated risk of infarcts.

Disadvantages would include estrogen-related effects on the uterus, vagina, breast tissue, and liver. An undesirable feature in this situation is that in the present state of the art no phytoestrogen is currently available that can be adapted for organ-selective prophylaxis or therapy in the presence of estrogen deficiency.

In view of this state of the art, the object of the present invention therefore is to make plant medications with estrogen-type effects available that demonstrate an organ-selective effect without or with only mild effects upon the uterus.

The solution to achieve this object is found through an application according to claim 1.

With the phytoestrogen-containing extracts of the present invention, particularly those from vitex agnus castus and preferably their fruits, it is possible for the first time to obtain a selective ER-beta effect without a uterotropic effect. Through the use of vitex agnus castus extracts it is possible to treat the following clinical situations and pathophysiological conditions through modulation of ER-beta:
Obesity and thereby to possibly influence the metabolic syndrome, particularly hypertension, arteriosclerosis, cardiac infarct, hyperandrogenemia;
Menopausal continence disorders;
Menopausal heat surges associated with hyperstimulation of the hypothalamic gonadotropin releasing hormone pulse generator;
Steroid hormonal synthesis disorders, particularly that of progesterone synthesis of the human corpus luteum, resulting in luteal insufficiency;
Alzheimer's disease associated with elevated expression of estrogen receptor beta in hippocampal neurons.

The extracts for adaptation according to the invention may be used in standard pharmaceutical formulations, for example in the form of drops, tablets, coated tablets, capsules, granulates, dragées, suppositories, ointments, creams, or similar.

It is preferable to manufacture extracts from vitex agnus castus, particularly its fruit and/or leaves, through ethanol extraction or through extraction with CO₂ from the plants or plant parts and use them for the application according to the invention.

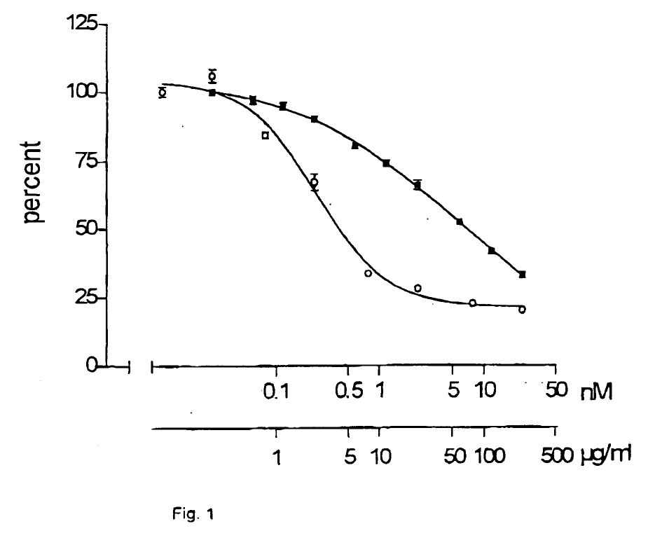

To demonstrate the presence of phytoestrogens in vitex agnus castus (VAC) extracts, the inventors have used a competitive estrogen receptor assay in which radiolabeled estradiol is displaced from the receptors through ligands that likewise bind to the receptors, hereafter called receptor modulators. On the one hand, non radiolabeled estradiol and on the other hand the investigational extracts were used as receptor modulators. The receptor preparation consisted of the cytosolic fraction of porcine uteri, which contains both estrogen receptor subtypes, ER-alpha and ER-beta. The results of a typical experiment are shown in FIG. 1.

FIG. 1 shows the displacement curves of estradiol (E₂, empty symbols) and VAC extracts (filled symbols). The ordinates give the percentage share of the (still remaining) bindings of radiolabeled estradiol on the estrogen receptors. The upper abscissa illustrates the E₂ concentration, while the lower abscissa provides the VAC concentration. The displacement obtained with the lowest concentration of the test substances was defined as 100%. The mean values±SEM were obtained from three measurements. The E₅₀ of the VAC is 7.8 μg/mL.

As FIG. 1 demonstrates, the bindings in the VAC extracts compete in a dose-dependent manner with radiolabeled estradiol for the binding sites of the estrogen receptors. The collected data significantly demonstrate that the investigational extracts contain phytoestrogen.

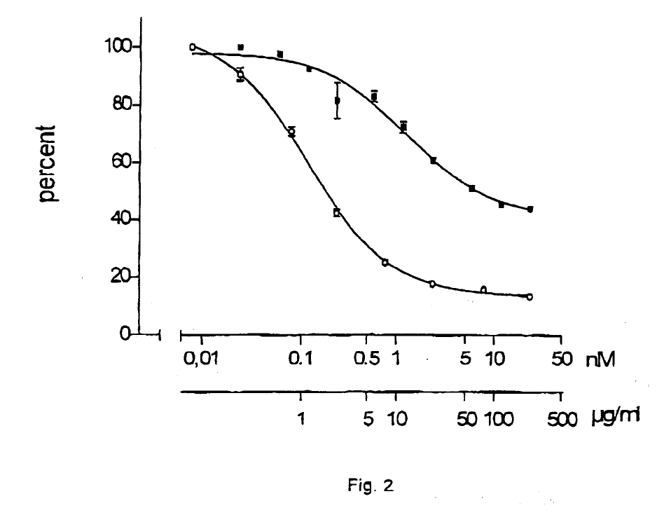

To further reinforce this finding, the same assay was performed with a receptor preparation from the human endometrium. Again, both ER subtypes are present in the solution. The results of these investigations are illustrated in FIG. 2.

FIG. 2 shows the displacement curves of radiolabeled estradiol from human endometrial ER through estradiol (E₂, empty symbols) and VAC extracts (filled symbols). The ordinates give the percentage share of (still remaining) bindings of radiolabeled estradiol on the estrogen receptors. The upper abscissa designates the E₂ concentration, while the lower abscissa gives the VAC concentration. The displacement obtained with the lowest concentration of the test substances was defined as 100%. The mean values±SEM were obtained from three measurements. The E₅₀ of the VAC was 12.7 μg/mL.

The results clearly indicate that phytoestrogens from VAC extracts bind to human estrogen receptors, which constitutes the molecular initial event in an estrogen-type effect of VAC binding in humans.

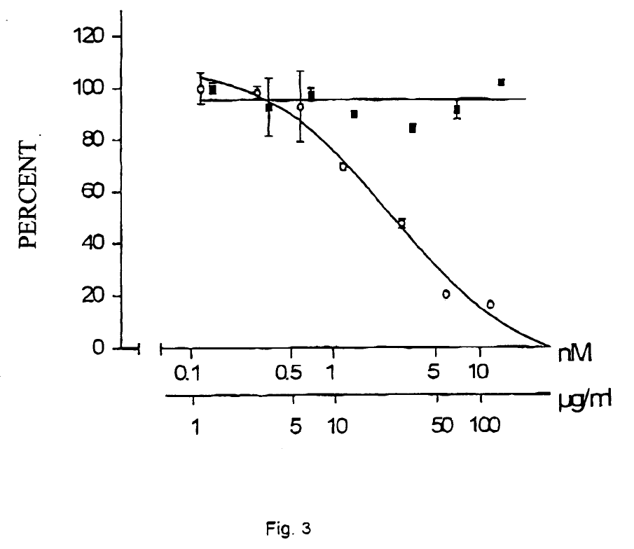

Because of the volatility of recombinant receptor proteins, subtype-specific ER assays (ER-alpha or ER-beta) can be performed. The result, in the form of a typical ER-alpha displacement curve, is illustrated in FIG. 3.

FIG. 3 shows the dose/effect curves of estradiol (E₂, empty symbols) and VAC extracts supplied by the applicant (filled symbols) in an assay using recombinant human ER-alpha. The upper abscissa gives the E₂ concentration, while the lower abscissa gives the VAC concentration. The binding with the lowest concentration of the test substances was defined as 100%. The mean values±SEM were obtained from three measurements per concentration value.

While E₂ demonstrates dose-dependent competition with the radiolabeled receptor ligands, the phytoestrogens of the VAC extract do not bind with ER-alpha. Therefore no E₅₀ can be calculated.

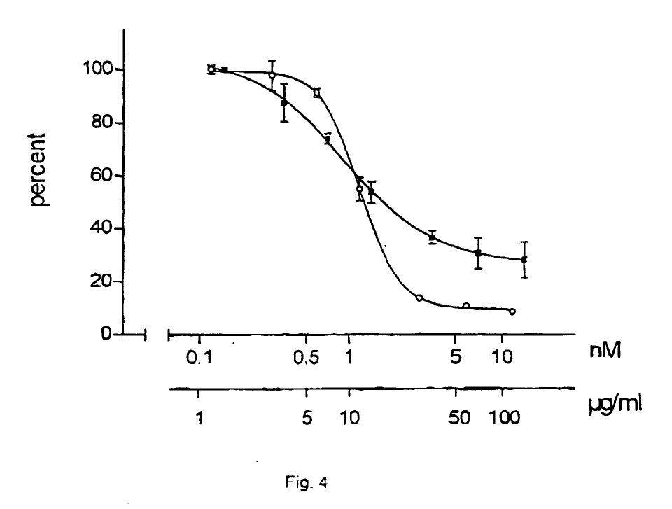

Then, using the same test bindings as in the ER-alpha assay, ER-beta assays were performed. The data and the graph of a representative experiment are given in FIG. 4.

FIG. 4 shows the dose/effect curves of estradiol (E₂, empty symbols) and VAC extracts supplied by the applicant (filled symbols) in an assay using recombinant human ER-beta. The upper abscissa gives the E₂ concentration, while the lower abscissa gives the VAC concentration. The binding with the lowest concentration of the test substances was defined as 100%. The mean values±SEM were obtained from three measurements per concentration value. The E₅₀ of the VAC was 9.9 μg/mL.

In contrast to the ER-alpha assay, the phytoestrogens of the VAC extracts supplied by the applicant bind in a dose-dependent manner with ER-beta. These data confirm without doubt the ER-beta selectivity of the vitex agnus castus extracts supplied by the applicant.

In what follows, the estrogenic effect of vitex agnus castus on human granulosa/luteal cells and porcine luteal cells is demonstrated.

The steroid secretion of the ovarian follicle and the corpus luteum is regulated endocrinically by the hypophysial hormones LH and FSH. In addition, a local control is performed through estradiol, which is designated as paracrine regulation. This local control requires the expression of the estrogen receptor. New molecular biology research suggests that granulosa cells express the ER-beta subtype in a very dominant manner compared with the ER-alpha. By contrast, luteal cells express ER-beta exclusively.

Luteal cell cultures are an established test method for the paracrine effect of steroids. Such cells are not available from humans, since all surgical procedures in which biopsies can be taken are always performed at the follicular phase to avoid the risk of an undetected pregnancy. However, these granulosa/luteal cells become available in the course of in vitro fertilization programs and are maintained in culture, and they behave very much like luteal cells in terms of receptor population and secretory function. An estrogenic effect of vitex agnus castus was tested with this human in vitro testing system.

Next, the effect of estradiol 17-beta (naturally occurring estradiol) on the progesterone secretion of human granulosa/luteal cells needed to be tested against that of the isomer estradiol 17-alpha.

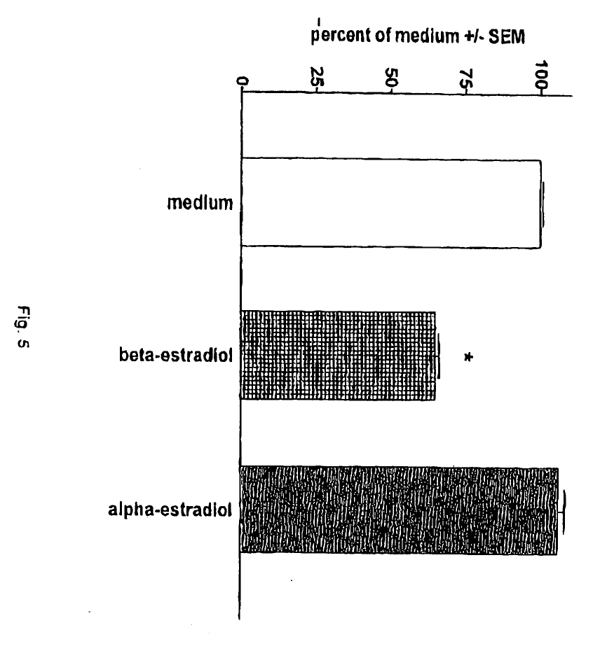

The data are illustrated in graphical form in FIG. 5.

FIG. 5: A 5-hour incubation of these cells with 17-beta estradiol (1 μM) produces a significant inhibition of progesterone secretion, while the isomer 17-alpha estradiol (likewise 1 μM) produces no effect. The * indicates a significant deviation from the (culture) medium control.

This means:
1. In vitro inhibition of progesterone secretion is an estrogenic effect.
2. The effect of estradiol 17-beta is highly specific, since 17-alpha estradiol cannot produce this effect.

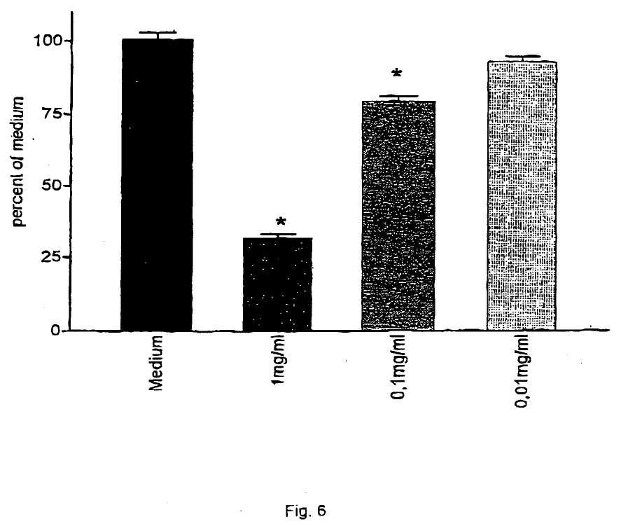

FIG. 6 summarizes the effect of vitex agnus castus extracts upon progesterone secretion under this testing system after a 5-hour incubation.

As FIG. 6 shows, the vitex agnus castus extract inhibits progesterone secretion of the human granulosa/luteal cells exactly like the naturally occurring 17-beta estradiol. This has to be considered pharmaceutically as an estrogenic effect. The * indicates a significant deviation from the control (=culture medium). The concentration data on the abscissa indicate the effective concentration of the VAC extract in the test sample.

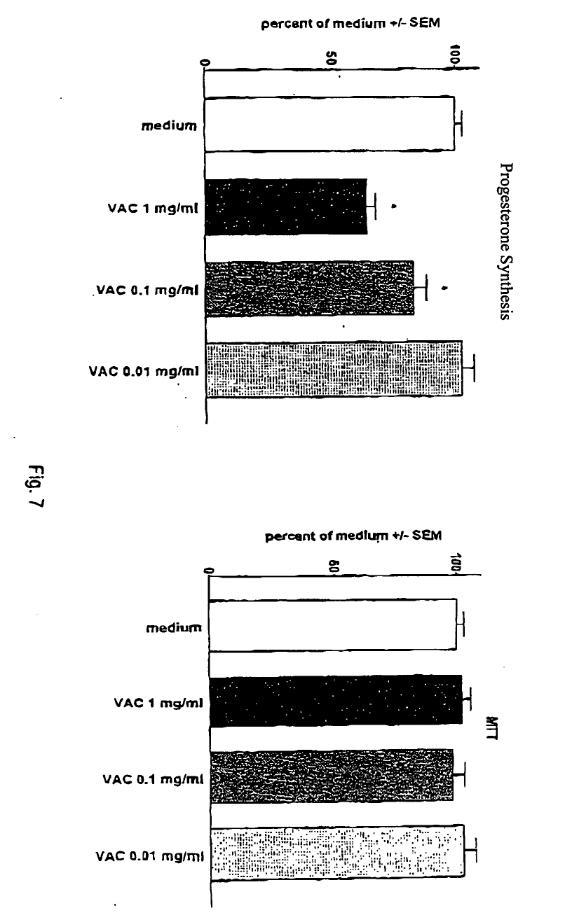

Since the human granulosa/luteal cells predominantly but not exclusively express ER-beta, the experiment was repeated using porcine luteal cells, i.e. cells with exclusively ER-beta expression. FIG. 7 illustrates the applicable results.

FIG. 7 shows that vitex agnus castus has an evident estrogenic effect on these cells also because progesterone secretion (left side of graphic, first three columns after the first medium control) is significantly inhibited. A nonspecific cytotoxic effect was excluded by measuring the cell vitality (through MTT staining using standard methods) (right side, last three columns after the second medium control). Cell vitality is not affected by the vitex agnus castus extract. This means that decreased progesterone secretion is a specific estrogenic effect.

The * indicates a significant deviation from the (culture) medium control. The concentration data on the abscissa indicate the effective concentration of the VAC extract.

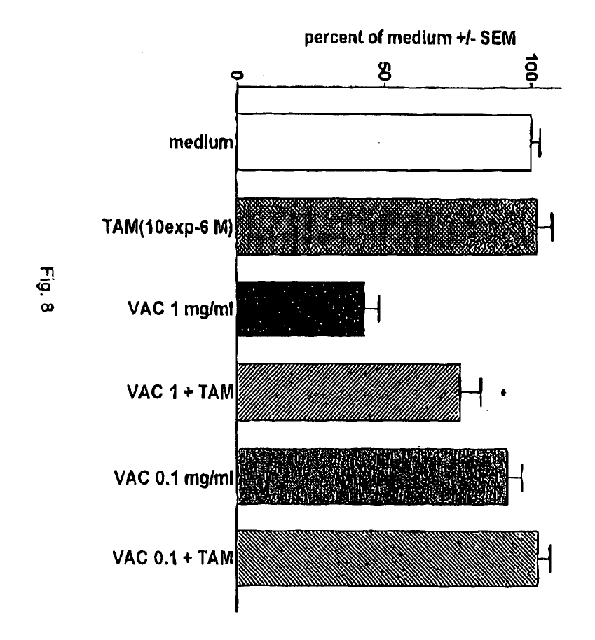

To reinforce this conclusion, the cells were incubated with vitex agnus castus extract along with the anti-estrogen tamoxifen. FIG. 8 illustrates the results in a bar diagram.

In FIG. 8, the * indicates a significant deviation from the (culture) medium control. The descriptions on the abscissa mean as follows: TAM=tamoxifen; the concentration data indicate the effective concentration of the VAC extract; and VAC+TAM indicates the combination of the VAC extract with a given concentration, from the last bar, of pure VAC extract plus tamoxifen.

FIG. 8 shows that the anti-estrogen tamoxifen is of itself without effect upon progesterone excretion. Through tamoxifen binding on the ER fewer binding sites become available for VAC components, and the elicited effect—inhibition of progesterone secretion—is reduced. This finding clearly indicates that the effect of VAC on progesterone synthesis is mediated in a specific manner via the estrogen receptors.

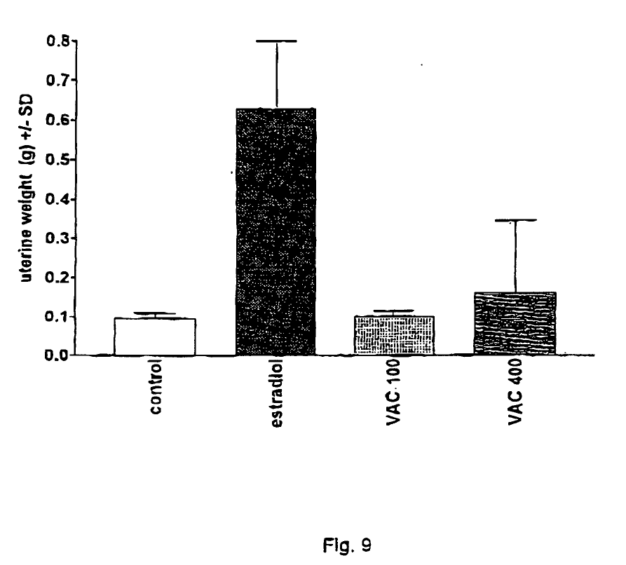

The following experiment was performed to test for uterotropic effect; the data are illustrated as a graph in FIG. 9.

Two different concentrations of VAC extracts (100 mg/kg body weight=VAC100 and 400 mg/kg body weight=VAC400) were added during a three-month period to the feed of ovarectomized female and orchiectomized male rats. In contrast to estradiol, VAC had no uterotropic effect and even the vaginal epithelium remained intact. Both proliferative effects of estradiol are undesirable in the context of peri- and post-menopausal estrogen replacement therapy or hormone replacement therapy in ovarectomized women since endometrial hyperplasia secondary to the persistent proliferative effect of estradiol cannot be excluded in the presence of chronic administration of estrogens and the absence of an opposing regulation factor through progestogens.

The uterine weight of the rats is listed on the ordinates in FIG. 9. The individual bars represent the control group (ovarectomized only), an estradiol positive control, and the effect with two different VAC extract concentrations.

In FIG. 9, a uterotropic effect similar to that of estradiol could not be detected in the VAC-treated animals. This means that VAC does not act proliferatively on the uterine tissue.

However, a surprising finding emerged from further investigations—that VAC can exercise estrogenic effects on other organ systems.

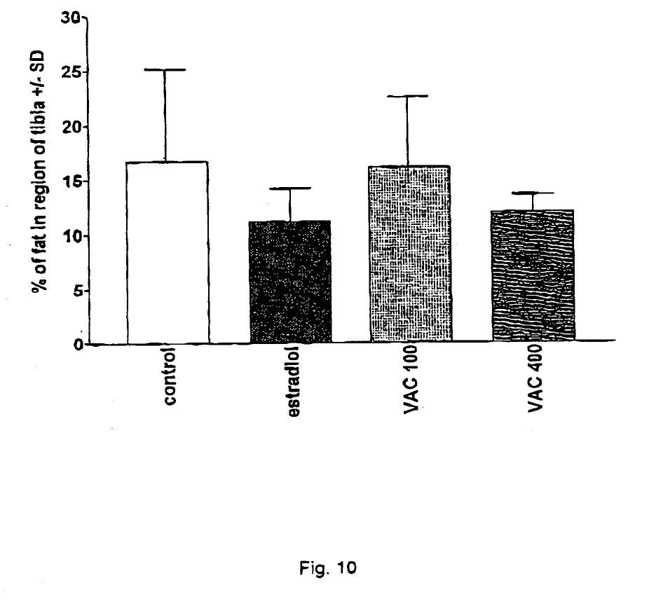

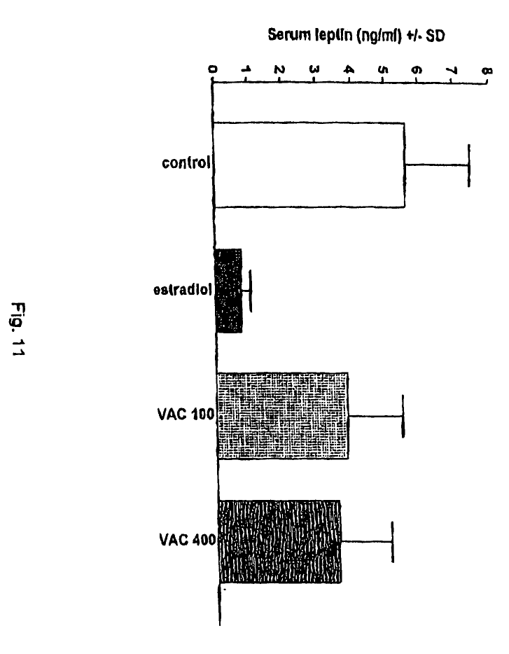

The abdominal and paratibial adipose tissue of male and female rats was examined with computer-assisted tomography. A decrease in both tissue segments was observed under E₂ and the high dose of vitex agnus castus. The serum leptin levels of the VAC-treated animals were concomitantly reduced. The results of this investigation are given in FIGS. 10 and 11.

FIG. 10 shows the effect of estradiol and two different VAC concentrations on the paratibial fatty tissue, represented as the percentage surface share of fatty tissue on a CT scan of the tibial area compared with a control.

FIG. 11 shows the effect of estradiol and two different VAC concentrations on the serum leptin level, represented on the ordinates in ng/mL.

As FIGS. 10 and 11 demonstrate, a decrease in paratibial fat content and a decrease in the serum leptin concentration occur under VAC administration.

Likewise, with computer-assisted tomography, the change in bone density (less osteoporotic development) was observed on the tibial metaphysis in male and female rats. It emerges that the bone density clearly declined less under VAC than it did in the untreated controls.

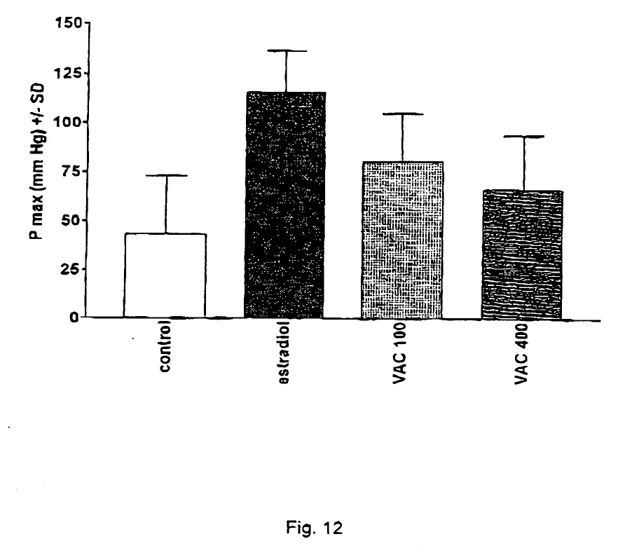

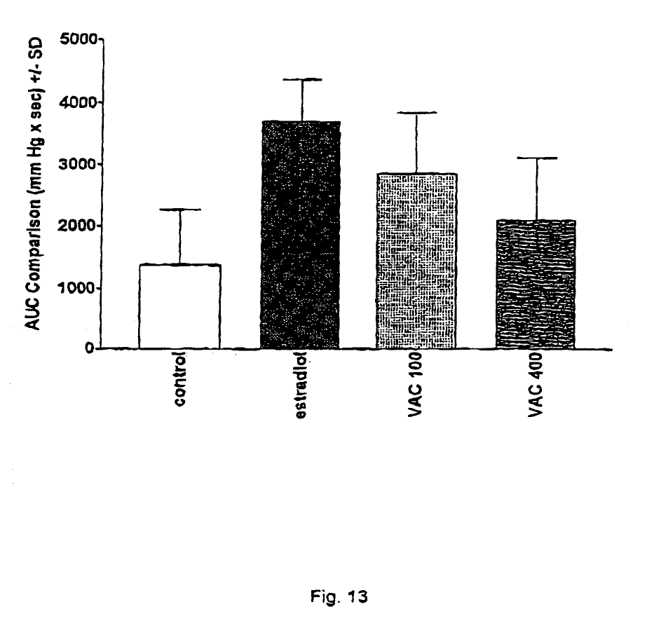

The instillation of 1 mL normal saline solution within 1 minute via a bladder catheter in ovarectomized female rats leads to an increase in internal bladder pressure. In animals treated with E₂ replacement therapies for three months, the pressure increase was three times higher than in the placebo-treated control animals, and was slightly weaker but still significant in estradiol-treated animals, when the animals were treated with VAC. The results of this investigation are given in FIGS. 12 and 13.

The maximum internal bladder pressure is given in mmHg on the ordinates of FIG. 12. It was measured for a control, for estradiol as a positive control, and for two different VAC concentrations.

By contrast, the ordinates in FIG. 13 show the pressure area under the curve (AUC) in mmHg×sec for a control, for estradiol as a positive control, and for two different VAC concentrations.

In view of this it is therefore possible to use VAC extracts for the manufacture of medications to treat:
Obesity and thereby possibly to influence the metabolic syndrome, particularly hypertension, arteriosclerosis, cardiac infarct, hyperandrogenemia;
Menopausal continence disorders;
Menopausal heat surges associated with hyperstimulation of the hypothalamic gonadotropin releasing hormone pulse generator;
Steroid hormonal synthesis disorders, particularly that of progesterone synthesis of the human corpus luteum, resulting in luteal insufficiency; and
Alzheimer's disease associated with elevated expression of estrogen receptor beta in hippocampal neurons.

### LITERATURE

1. Hrabovszky E, Shughrue P J, Merchenthaler I, Hajszan T, Carpenter CD, Liposits Z, Petersen SL. Detection of estrogen receptor-beta messenger ribonucleic acid and 1251-estrogen binding sites in luteinizing hormone-releasing hormone neurons of the rat brain. Endocrinology 2000 Sep., 141(9):3506-9.
2. Hosokawa K, Ottander U, Wahlberg P, Ny T, Cajander S, Olofsson I J. Dominant expression and distribution of oestrogen receptor beta over oestrogen receptor alpha in the human corpus luteum. Mol Hum Reprod 2001 Feb., 7 (2):137-45.
3. Taylor A H, Al-Azzawi F. Immunolocalisation of oestrogen receptor beta in human tissues. J Mol Endocrinol 2000 Feb., 24(1):145-55.
4. Kuiper G G, Carlsson B, Grandien K, Enmark E, Haggblad J, Nilsson S, Gustafsson J A. Comparison of the ligand binding specificity and transcript tissue distribution of estrogen receptors alpha and beta. Endocrinology 1997, 138:863-870.
5. Saunders P T, Maguire S M, Gaughan J, Millar M R. Expression of oestrogen receptor beta (ER beta) in multiple rat tissues visualized by immunohistochemistry. J Endocrinol 1997, 154:R13-R16.
6. Mãkelä S, Strauss L, Kuiper G, Valve E, Salmi S, Santti R, Gustafsson J A. Differential expression of estrogen receptors alpha and beta in adult rat accessory sex glands and lower urinary tract. Mol Cell Endocrinol 2000, 164:109-116.

1-3. (canceled)

4. (canceled)

5-7. (canceled)

8. A method for treating clinical situations and pathophysiological conditions comprising the step of administering a phytoestrogen-containing extract, wherein said phyto-estrogen containing extract selectively modulates the estrogen receptor beta without producing a uterotropic effect, and wherein the clinical situations and pathophysiological conditions are selected from the group consisting of obesity; menopausal continence disorders; menopausal heat surges associated with hyperstimulation of the hypothalamic gonadotropin releasing hormone pulse generator; steroid hormonal synthesis disorders; and Alzheimer's disease associated with elevated expression of estrogen receptor beta in hippocampal neurons.

9. The treatment method of claim 8 wherein the clinical situations and pathophysiological conditions is obesity and the treatment influences the metabolic syndrome, thereby influencing the cause of hypertension.

10. The treatment method of claim 8 wherein the clinical situations and pathophysiological conditions is obesity and the treatment influences the metabolic syndrome, thereby influencing the cause of arteriosclerosis.

11. The treatment method of claim 8 wherein the clinical situations and pathophysiological conditions is obesity and the treatment influences the metabolic syndrome, thereby influencing the cause of cardiac infarct.

12. The treatment method of claim 8 wherein the clinical situations and pathophysiological conditions is obesity and the treatment influences the metabolic syndrome, thereby influencing the cause of hyperandrogenemia.

13. The treatment method of claim 8 wherein the steroid hormonal synthesis disorders is that of progesterone synthesis of the human corpus luteum, resulting in luteal insufficiency.

14. The use of claim 8 wherein the phytoestrogen-containing extract is an ethanol or CO₂ extract from the vitex agnus castus.

15. The treatment method of claim 8 wherein the phytoestrogen-containing extract is a standard pharmaceutical formulation.

16. The use of claim 15 wherein the phytoestrogen-containing extract is an ethanol or CO₂ extract from the vitex agnus castus.

17. The treatment method of claim 15 wherein the pharmaceutical formulation of the phytoestrogen-containing extract is selected from the group consisting of drops, tablets, coated tablets, capsules, granulates, dragées, suppositories, ointments and creams.

18. The use of claim 17 wherein the pharmaceutical formulation is an ethanol or CO₂ extract from the vitex agnus castus.
